(19) United States
(12) Patent Application Publication
(10) Pub. No.: US 2022/0072293 A1
(43) Pub. Date: Mar. 10, 2022

(54) DRUG DELIVERY DEVICE USING ULTRASONIC WAVE

(71) Applicant: Sanghyun Chang, Gyeonggi-do (KR)

(72) Inventor: Sanghyun Chang, Gyeonggi-do (KR)

(21) Appl. No.: 17/446,306

(22) Filed: Aug. 30, 2021

(30) Foreign Application Priority Data
Sep. 10, 2020 (KR) 10-2020-0115884

(51) Int. Cl. A61M 37/00

(52) U.S. Cl. CPC A61M 37/0092; A61M 2037/0007

(57) ABSTRACT

A drug delivery device using ultrasonic wave according to an embodiment of the present invention includes: an ultrasonic wave generating device for generating ultrasonic wave by receiving the alternating current electric energy generated from an external alternating current power supply unit; and a drug injection device configured to be coupled with and separated from the ultrasonic wave generating device and to inject a drug into the human body by using the ultrasonic wave energy transmitted from the ultrasonic wave generating device, wherein the ultrasonic wave generating device includes an ultrasonic wave generating portion for converting alternating current electric energy into ultrasonic wave and a medium portion for serving to transfer the ultrasonic wave energy generated by the ultrasonic wave generating portion to the drug accommodated in the drug injection device, and wherein the drug injection device includes a drug injection port capable of injecting the drug present in a separate container into the drug injection device.

### BACKGROUND OF THE INVENTION

### 1. Field of the invention

The present invention relates to an apparatus for injecting a drug into the human body without a needle by using ultrasonic wave.

### 2. Description of the Prior Art

The development of painless injection needles (painless syringes) is a global trend, and major medical device manufacturers at home have developed injection needles with a diameter of 0.18 millimeters capable of injecting drugs and have started to be exported to Europe.

Many researchers and technicians around the world are constantly working on painless needles capable of injecting drugs. In the meantime, special techniques such as laser processing, etc., have been successfully used to manufacture 0.1 millimeter-sized needles (capable of collecting blood or injecting drugs) now.

Meanwhile, methods of performing a procedure on the skin in various forms to prevent skin aging have been introduced.

Among the methods, the most representative method for massaging the skin with beneficial substances containing nutrients has been most widely used.

In other words, in recent years, various procedures for supporting beauty or health have been performed, out of which a procedure for simultaneously injecting many drugs into a large area is widely used.

When using a conventional syringe in the procedure of injecting a drug into such a large area, an injection needle pierces a recipient's skin at every injection, and thus the recipient must feel pain each time, while an operator must put the injection needle at an appropriate depth, thus providing a disadvantage of causing a lot of time and effort for the procedure.

A syringe capable of simultaneously injecting into many areas has been developed by addressing the above disadvantages, and is disclosed in Korean Registered Patent No. 1066883 as an example.

The prior patent describes a syringe which includes: a hollow cylindrical body; an injection needle hub tightly coupled to a rear end of the cylindrical body; a plurality of injection needles arranged at a predetermined distance from each other inside the cylindrical body, having a tip protrude more than the tip of the cylindrical body, and having the rear end arranged so that the drug injected through the injection needle hub is introduced; a first electrode which is received inside the cylindrical body and comes into contact with a part of the plurality of injection needles; and a second electrode which is received inside the cylinder and comes into mutual contact with the remaining part of the plurality of injection needles.

Such a conventional injection apparatus or device having a multi-needle has the advantage of injecting into a large at one time.

Meanwhile, a microneedle therapy system (MTS), also called a fractional microneedle therapy system or a roller needle, uses micro-needling (micro-needles) to create hundreds of thousands of micro-holes without damaging the skin to induce drug absorption and stimulate the skin, thereby helping natural regeneration of the skin.

The MTS treatment is a treatment method that allows an ampoule effective for acne, whitening and skin regeneration to be absorbed into the dermis layer of the skin, and is effective in treating acne and removing acne scars. And, the MTS treatment is also a treatment method that utilizes 100% of the skin regeneration rate to enable natural healing without damaging the skin.

The prior art of the MTS procedure has a problem in that the absorption rate of the drug into the skin is remarkably low and the procedure is inconvenient in such a way that the skin is pierced with dozens of needles while rolling, and then the drug is separately applied and permeated by massage.

To solve this problem, a drug delivery system has been developed to deliver drugs directly to a desired site with dozens of painless syringes.

### SUMMARY OF THE INVENTION

The present invention proposes a device capable of stably administering a drug into the human body through ultrasonic wave while replacing a conventional syringe using a needle.

A drug delivery device using ultrasonic wave according to an embodiment of the present invention includes: an ultrasonic wave generating device for generating ultrasonic wave by receiving the alternating current electric energy generated from an external alternating current power supply unit; and a drug injection device configured to be coupled with and separated from the ultrasonic wave generating device and to inject a drug into the human body by using the ultrasonic wave energy transmitted from the ultrasonic wave generating device, wherein the ultrasonic wave generating device includes an ultrasonic wave generating portion for converting alternating current electric energy into ultrasonic wave and a medium portion for serving to transfer the ultrasonic wave energy generated by the ultrasonic wave generating portion to the drug accommodated in the drug injection device, and wherein the drug injection device includes a drug injection port capable of injecting the drug present in a separate container into the drug injection device.

And, the medium portion is disposed on a movement path of the ultrasonic wave generated by the ultrasonic wave generating portion and includes any one of water, gel or oil as a material for delivering the ultrasonic wave to the drug serving as a target.

And, the drug injection device includes: a focusing housing formed in a conical shape while forming therein a drug storage portion corresponding to a space having the drug stored therein; a separation membrane disposed at one side of the focusing housing to prevent a material constituting the medium portion from flowing out when the medium portion is combined; an input portion integrally formed with the focusing housing and having a cross section smaller than a cross section of the focusing housing so that a high pressure is applied to a drug movement by the ultrasonic wave energy; and a human body input surface for discharging the drug while forming an end of the input portion.

And, the focusing housing is configured to have a conical shape of which a cross section is gradually reduced from the ultrasonic wave generating device toward the input portion so as to provide an inclined surface when viewed in a cross section, and the inclined surface is formed such that an incident angle, at which the ultrasonic wave generated from the ultrasonic wave generating device is incident on the inclined surface of the focusing housing, exceeds 45°.

And, one surface of the ultrasonic wave generating portion includes a curved surface formed to provide a predetermined curvature.

The device proposed has an advantage of significantly reducing a contamination with bacteria/germs during the process of injecting a drug into the human body, and also providing a simple device to be easily used.

In particular, the device has no needle and thus can stably inject the drug into the human body without pain, thereby providing an advantage of being available to various age groups.

### BRIEF DESCRIPTION OF THE DRAWINGS

FIG. 1 is a view for explaining a sound wave when an alternating current (AC) signal is applied to an ultrasonic wave generator.

FIG. 2 is a view for explaining the detailed configuration of a drug delivery device using ultrasonic wave according to an embodiment of the present invention.

FIG. 3 is a view for explaining the shape of an ultrasonic wave generating portion according to an embodiment of the present invention.

FIG. 4 is a view for explaining the design of an outer circumferential surface angle of a focusing housing according to an embodiment of the present invention.

FIG. 5 is a view for showing the separation and coupling of the ultrasonic wave generator and a cartridge according to an embodiment of the present invention.

### DETAILED DESCRIPTION OF THE EXEMPLARY EMBODIMENTS

Hereinafter, the embodiments of the present invention will be described in detail with reference to the accompanying drawings. However, the scope and idea of the embodiments of the present invention may be determined from the description of the embodiments of the present invention, and the inventive idea according to the embodiments of the present invention may include the modifications, such as addition, deletion, change, etc. of components with respect to the proposed embodiments.

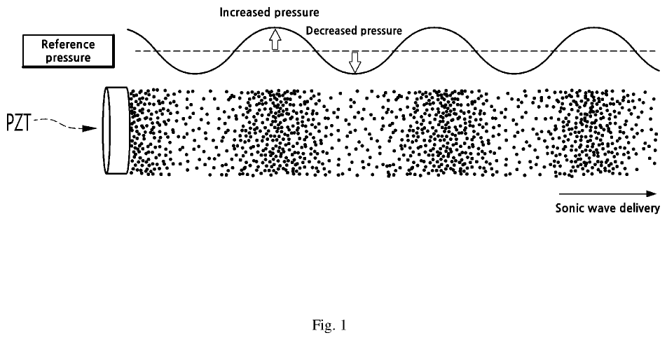

FIG. 1 is a view for explaining a sound wave when an alternating current (AC) signal is applied to an ultrasonic wave generator.

When an AC electric signal is applied to PZT, one of the ultrasonic wave generators, the PZT may generate an inverse piezoelectric effect so as to form a surface vibration. Due to the vibration, the sound wave may be transmitted to a medium coming into contact with the PZT.

The present invention is to propose a device capable of injecting a drug into the human body under pressure due to the vibration caused by transmitting the ultrasonic wave to the medium.

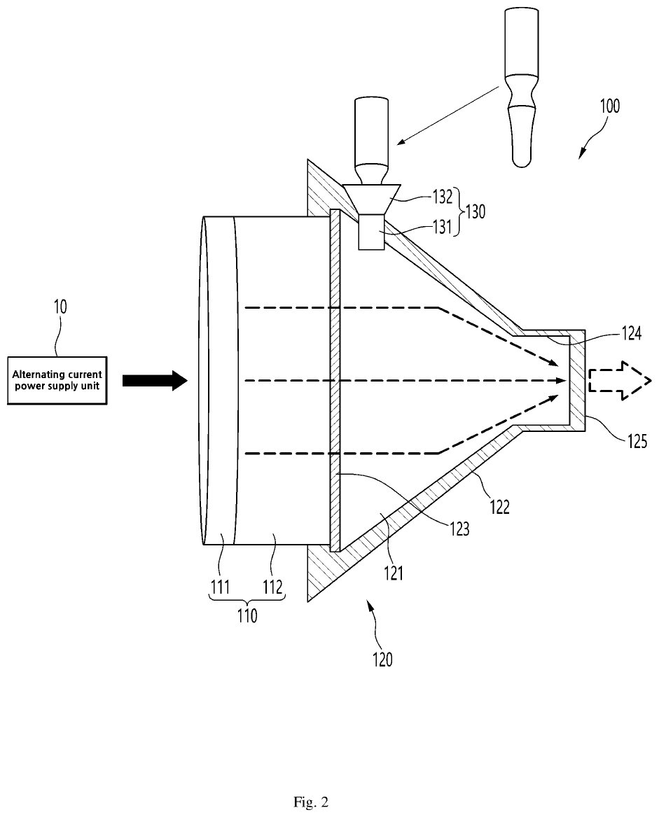

FIG. 2 is a view for explaining the detailed configuration of a drug delivery device using ultrasonic wave according to an embodiment of the present invention.

Referring to FIG. 2, a drug delivery device 100 of an embodiment may include: an ultrasonic wave generating device 110 for generating ultrasonic wave by receiving the alternating current electric energy generated from an external alternating current power supply unit 10; and a drug injection device 120 configured to be detachably coupled to the ultrasonic wave generating device 110 and deliver the ultrasonic wave energy transmitted from the ultrasonic wave generating device 110 to a drug, which is to be injected into the human body.

In detail, the ultrasonic wave generating device 110 may include an ultrasonic wave generating portion 111 for converting alternating current electric energy into ultrasonic wave and a medium portion 112 for serving to transfer the ultrasonic wave energy generated by the ultrasonic wave generating portion 111 to the drug accommodated in the drug injection device 120.

The ultrasonic wave generating portion 111 may be made of a PZT element for converting alternating current electric energy into ultrasonic wave energy, and a shape thereof may be formed in a flat plate shape when there is a device for focusing energy from the outside, and may be also formed in a conical focusing shape in which the PZT element per se may focus energy to one point.

The medium portion 112 may be disposed on a movement path of the ultrasonic wave generated by the ultrasonic wave generating portion 111 and may refer to a material for delivering the ultrasonic wave to the drug serving as a target. For example, the medium portion 112 may include water, gel, various oils, etc.

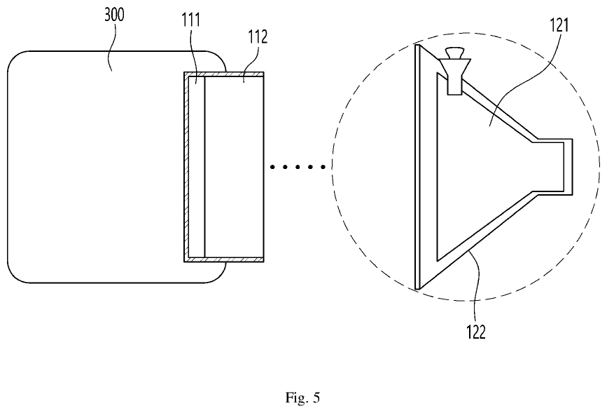

The medium portion 112 may be filled in the entire or partial region of the inner space of a medium portion frame 113 (see FIG. 5). The dissolved gas in solution may reduce the strength of cavitation, and thus the medium portion 112 may be used in a degassed form in which all of the dissolved gas is removed. The medium portion frame 113 constituting the medium portion 112 may be made of a common material that does not change the physical properties of the medium portion 112.

Ultrasonic wave may be sound wave. Thus, unlike light, the ultrasonic wave may require the medium portion 112 so as to be propagated and a transmission speed may vary depending on the properties of the material of the medium portion.

The transmission efficiency of the power wirelessly transmitted between the ultrasonic wave generating portion 111 and the drug injection device 120 may vary depending on the properties of the medium portion, that is, the material of the medium portion, geometry, a transmission medium, attenuation and a distance between the ultrasonic wave generating portion 111 and the drug injection device 120.

In one embodiment of the present invention, in addition to the gel, water and oil described above, the medium portion 112 may be prepared from a silicon-based material such as silicon rubber having an acoustic impedance similar to that of human skin or blood, that is, an acoustic impedance of about 1.48 to 1.6 Mryl, or a high molecular compound including polymer such as epoxy, polyurethane or the like.

The acoustic impedance of silicone rubber may be about 1.44 to 1.46 Mryl, the acoustic impedance of epoxy may be about 1.52 Mryl, and the acoustic impedance of polyurethane may be about 1.55 to 1.56 Mryl.

Meanwhile, when describing the drug injection device 120 in more detail, the drug injection device 120 may include: a focusing housing 122 formed in a conical shape while forming a space of a drug storage portion 121 to accommodate/store a drug therein; a separation membrane 123 disposed at one side of the focusing housing 122 to prevent a material constituting the medium portion from flowing out when the medium portion 112 is combined; an input portion 124 integrally formed with the focusing housing 122 and having a cross section smaller than a cross section of the focusing housing 122 so that a high pressure is applied to a drug movement by the ultrasonic wave energy; a human body input surface 125 for discharging the drug moved under high pressure while forming an end of the input portion 124; and a drug injection port 130 communicating the drug storage portion 121 with the outside so as to inject the drug into the drug storage portion 121.

In detail, the focusing housing 122 may be made of a synthetic resin that is the same material as that of a general syringe including a needle, and may be detachably coupled to the ultrasonic wave generating device 110, in which a member for enabling separation and coupling may be formed at an end of the ultrasonic wave generating device 110 or the focusing housing 122.

The focusing housing 122 may have a conical shape of which a cross section gradually decreases from one side to the other, and the focusing housing 122 may have a drug storage portion 121, a space in which the drug can be stored/accommodated.

In addition, a through hole may be formed in a portion of the focusing housing 122, and a drug injection port 130 through which a drug may be supplied from an external drug container may be coupled to the through hole. The drug injection port 130 may include an injection port body 131 inserted into the through hole of the focusing housing 122, and a funnel 132 formed at an upper side of the injection port body 131.

The medical staff may insert the drug container accommodating the drug to be injected into a patient's body into the drug injection port 130 and store the drug in the drug storage portion 121.

The separation membrane 123 may serve to transmit the ultrasonic wave energy emitted from the ultrasonic wave generating device 110 into the drug storage portion 121 while serving as a partition to prevent the drug contained in the drug storage portion 121 from flowing out, and the separation membrane 123 may be made of, for example, a film.

The input portion 124 formed at an end side of the focusing housing 122 may be a member for applying a high pressure to the drug moving to a human body input surface 125 by ultrasonic wave energy, and may have a cross section thereof formed to be smaller than a cross section of the focusing housing 122.

The separation membrane 123, the focusing housing 122, the input portion 124, and the drug injection port 130 may be collectively referred to as a cartridge, and the cartridge may be configured to be separated and replaced from the ultrasonic wave generating device 110 after a single drug injection and a single drug input into the human body.

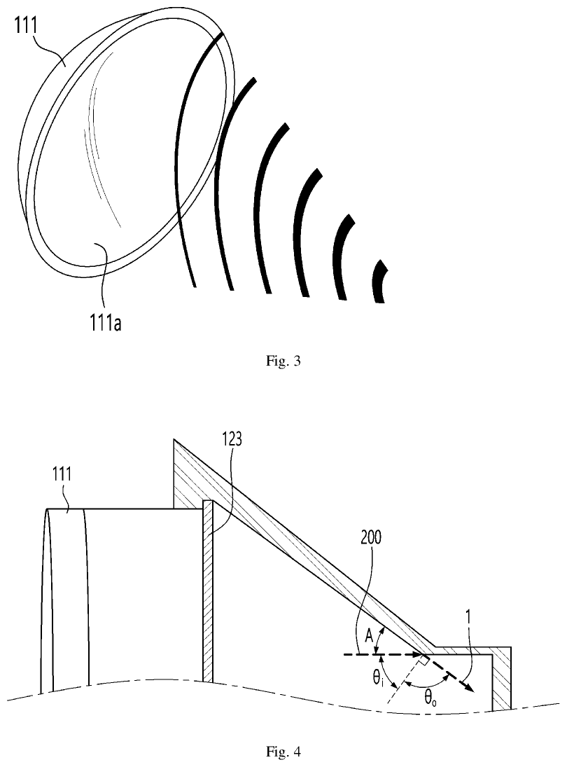

FIG. 3 is a view for explaining the shape of an ultrasonic wave generating portion according to an embodiment of the present invention.

Referring to FIG. 3, the ultrasonic wave generating portion 111 of an embodiment may be formed in a focusing type, and one surface facing the drug injection device 120 may include a curved surface 111a formed to have a predetermined curvature.

In this case, the ultrasonic wave energy generated by the ultrasonic wave generating portion 111 may be emitted while being focused to the center of the curved surface 111a, and thus when the drug is located and the human body input surface is disposed in a corresponding focusing direction, the drug may be efficiently injected into the human body.

FIG. 4 is a view for explaining the design of an outer circumferential surface angle of a focusing housing according to an embodiment of the present invention.

Referring to FIG. 4, when viewed in a cross section, the focusing housing 122 of an embodiment may have the inclined surface of the focusing housing 122 formed such that the incident angle of ultrasonic wave exceeds 45° with respect to the inclined surface of the focusing housing 122.

In other words, in order to provide a total reflection when the ultrasonic wave energy generated by the ultrasonic wave generating portion 111 is incident on the inclined surface of the focusing housing 122, an incident angle B with respect to the inclined surface of the focusing housing may be formed to exceed a critical angle of 45°, which is designed to consider the drug substance and the material of the focusing housing.

According to Snell's law, the conditions under which the total reflection of ultrasonic wave energy is performed in the embodiment of the present invention are confirmed as follows.

Sin(θₒ)/Sin(θᵢ)=Co/Ci [Equation 1]

Here, in order to achieve a total reflection, Sin(θₒ)=1 is required and Sin(θᵢ)=Ci/Co (sonic wave velocity in drug/sonic wave velocity in focusing housing material) is required. Assuming that the sonic wave velocity in drug is approximately 1,480 m/s, the sonic wave velocity in the focusing housing may be 2,100 m/s. Thus, substituting the same into the equation, the result may be 1480/2100=0.705.

In other words, the result may be Sin(θᵢ)=0.705, and thus Arcsin (0.705) may become approximately 45°, and thus an inclined surface of the focusing housing may be designed so that the incident angle exceeds 45°. In other words, when an acute angle A formed by the inclined surface of the focusing housing with the incident direction of ultrasonic wave energy is 45° or less, the total reflection may be achieved toward the input portion 124 without loss due to reflection of ultrasonic wave energy.

FIG. 5 is a view for showing the separation and coupling of the ultrasonic wave generating device and a cartridge according to an embodiment of the present invention.

The ultrasonic wave generating device 110 may be repeatedly used while being coupled to a separate storage housing 300, and the medium portion 112 may be fixed and supported by the medium portion frame 113 or the medium portion 112 may be accommodated/stored in the medium portion frame 113 as described above.

As described above, the cartridge may be the drug injection device 120 including the focusing housing 122, the separation membrane 123, the input portion 124 and the drug injection port 130 as described above, and may be separated and disused from the ultrasonic wave generating device 110 for hygiene after storing the drug and completely injecting the drug into the human body.

What is claimed is:

1. A drug delivery device using ultrasonic wave comprising:
an ultrasonic wave generating device configured to generate ultrasonic wave by receiving alternating current electric energy generated from an external alternating current power supply unit;
and a drug injection device configured to be coupled with and separated from the ultrasonic wave generating device and to inject a drug into the human body by using ultrasonic wave energy transmitted from the ultrasonic wave generating device,
wherein the ultrasonic wave generating device includes an ultrasonic wave generating portion configured to convert alternating current electric energy into ultrasonic wave and a medium portion configured to deliver the ultrasonic wave energy generated by the ultrasonic wave generating portion to the drug accommodated in the drug injection device,
and wherein the drug injection device includes a drug injection port that allows the drug present in a separate container to be injected into the drug injection device.

2. The drug delivery device of claim 1, wherein the medium portion is disposed on a movement path of the ultrasonic wave generated by the ultrasonic wave generating portion and comprises any one of water, gel or oil as a material for delivering the ultrasonic wave to the drug serving as a target.

3. The drug delivery device of claim 1, wherein the drug injection device comprises: a focusing housing formed in a conical shape while forming therein a drug storage portion corresponding to a space having the drug stored therein; a separation membrane disposed at one side of the focusing housing to prevent a material constituting the medium portion from flowing out when the medium portion is combined; an input portion integrally formed with the focusing housing and having a cross section smaller than a cross section of the focusing housing so that a high pressure is applied to a drug movement by the ultrasonic wave energy; and a human body input surface configured to discharge the drug while forming an end of the input portion.

4. The drug delivery device of claim 3, wherein the focusing housing is configured to have a conical shape of which a cross section is gradually reduced from the ultrasonic wave generating device toward the input portion so as to provide an inclined surface when viewed in a cross section, and
the inclined surface is formed such that an incident angle, at which the ultrasonic wave generated from the ultrasonic wave generating device is incident on the inclined surface of the focusing housing, exceeds 45°.

5. The drug delivery device of claim 4, wherein one surface of the ultrasonic wave generating portion comprises a curved surface formed to provide a predetermined curvature.
